(19) United States
(12) Patent Application Publication
(10) Pub. No.: US 2002/0184655 A1
(43) Pub. Date: Dec. 5, 2002

(54) METHODS FOR THE DEGRADATION AND DETOXIFICATION OF ORGANIC MATERIAL USING URINE PRODUCED BY TRANSGENIC ANIMALS AND RELATED TRANSGENIC ANIMALS AND PROTEINS

(76) Inventors: HENRYK LUBON, ROCKVILLE, MD (US); REKHA PALEYANDA, GAITHERSBURG, MD (US); WILLIAM DROHAN, SPRINGFIELD, VA (US); WILLIAM VELANDER, BLACKSBURG, VA (US)

Correspondence Address: FOLEY & LARDNER, 3000 K Street Suite 500, Washington, DC, 20007-5109, US

(21) Appl. No.: 08/982,284

(22) Filed: Dec. 1, 1997

(51) Int. Cl. A01K 67/027; A01K 67/00

(52) U.S. Cl. 800/4; 800/8; 800/14; 800/18; 800/24

(57) ABSTRACT

A method of producing a protein that degrades or detoxifies organic material is described. This method involves producing a non-human transgenic animal that produces such protein in its urine, and has stably integrated into its genome an exogenous gene encoding a protein that is detectable in the urine. Thus, a non-human transgenic animal that produced such protein in its urine, and a method of degrading or detoxifying organic materials also is described. Also a facility comprising a non-human transgenic animal that produce in its urine a protein that degrades or detoxifies organic material and a structure containing such animal is described. A method of altering a substance naturally found in urine is described. A DNA construct used in producing the non-human transgenic animal also is described.

### BACKGROUND OF THE INVENTION

1. Field of the Invention

This invention applies technological advancements in the field of transgenics to manage animal, plant, industrial and agricultural related wastes that are potential environmental pollutants.

### Animal Urine and Waste

Animals excrete a variety of nitrogen waste products like guanine, creatine, creatinine, amino acids and trimethylamine oxide, but ammonia, urea and uric acid predominate. Nitrates and microorganisms from animal wastes have the potential to contaminate groundwater. Storm runoff can transport manure to surface waters. High ammonia levels in surface water are toxic to fish and other aquatic fauna, while excessive nitrates and nitrites can be toxic to animals. Nitrates from animal waste or chemical fertilizers can lead to infant illness and death, from nitrate-induced methhemoglobinemia. Pathogenic microbes can make water unfit for livestock or human consumption. Health risks include salmonellosis, antibiotic resistance in microbes, toxic residues from therapeutic and prophylactic agents administered to animals. Excess nutrients promote eutrophication, or increased algae or plant growth in a water supply. When the plants die, their decomposition by microorganisms depletes the dissolved oxygen in water, resulting in fish kills (Pork Industry Handbook).

Large quantities of food processing, crop, forestry, and animal solid wastes are generated in the United States each year. The major components of these wastes are biodegradable. However, they also contain components such as nitrogen, phosphorus, human and animal pathogens, medicinals, feed additives, salts, oxidation-demanding organic compounds and certain heavy metals, that under uncontrolled conditions can be detrimental to aquatic, plant, animal, or human life. Manure or urine and feces from poultry and livestock production is a major source of environmental pollution. For example, the national production of broilers and mature chickens was 5.6 billion, 242 million turkeys, 31 million ducks, and 69 trillion table eggs in 1989 based on the USDA. Annual production of fecal waste from poultry flocks was 8.8 million tons on a dry weight basis plus more than 106, 000 metric tons of broiler hatchery waste. Add to this the 37 million dead and condemned birds (Pope, C. W., Poultry Sci., 70: 1123-1125, 1990).

About 80% of nitrogen and phosphorus, and 90% of potassium in animal feed is excreted by monogastric animals like pigs and poultry. Phosphorus in manure is mainly in the organic form and is released slowly causing environmental problems in areas of intensive livestock production, while potassium is an inorganic salt that leaches readily and is available for plant use. Chemicals may be added to manure to immobilize nitrogen and phosphorus. Changing the feed by adding phytase enzyme to release unavailable phosphorus can reduce the manure content by 25-40%. Phytases belong to the family of histidine acid phosphatases and catalyze the hydrolysis of phytate, the major storage form of phosphate for plant seeds into inorganic phosphate, inositol, and inositol mono- to pentaphosphates. A phytase-encoding gene from the fungus Aspergillus fumigatus was overexpressed in A. niger (Pasamontes et al., Appl. Environ Microbiol. 63: 1696-1700, 1997). The substrate specificity of the phyA enzyme resembled that of the phytases of A. niger T213, A. terreus 9A1, M. thermophila, and Aspergillus ficuum. The enzyme is resistant to high temperatures and enzymatic activity occurs over a broad pH range. The nitrogen content of manure can be reduced from 22 to 41% by reducing protein levels and substituting specific amino acids.

Anaerobic bacteria grow in extreme environmental conditions and their enzymes have applications in organic waste treatment systems, as well as chemical and fuel production systems based on biomass-derived substrates or syngas. They provide catabolic enzymes for organic compounds that cannot be digested by enzymes of eukaryotic origin. They are needed for the catabolism of cholesterol, bile acids and steroid hormones; they hydrolyze several flavonoid glycosides to anticarcinogens and detoxify certain carcinogens. Industrially, anaerobic enzymes are used in the production of cheese, the conversion of starch to sweeteners, and the transformation of sawdust, wood chips and waste paper into fuel (Bokkenheuser, Clin. Infect. Dis. 16: S427-434, 1993). Specific enzymes from pathogenic soil microorganisms can convert urea, creatinine, uric acid, guanidino derivatives, and other non-protein nitrogen compounds (NPN). The enzymes utilize ammonia, potassium, phosphorus, and other potentially dangerous factors. For example, both aerobic and anaerobic bacteria can accumulate polyphosphate from waste. Bacteria belonging to the genus Acinetobacter, such as A. Johnsonii 210A, occur in a wide variety of activated sludges, in which enhanced biological phosphate removal is observed (Kortstee et al., FEMS Microbiol. Rev. 15: 137-153, 1994). Other poly-phosphate accumulating microorganisms may also be involved in phosphorus removal. Bacteria that accumulate polyphosphate and also denitrify will have implications in wastewater treatment.

Over 50% of municipal waste is paper. Cellulosic materials in forage and feces waste may be degraded by cell-free enzymes like cellulases from thermophilic fungi like Thermomonospora curvata, that are active at composting temperatures of around 65° C and by cellulolytic fungi of the genus Trichoderma. Acid pretreatment of cellulosic wastes can improve susceptibility to Fusarium acuminatum enzymes like avicelase, carboxymethycellulase, β-glucosidase, xylanase and pectinase. Other cellulase genes, such as the celA, celB, celF genes from Clostridium cellulolyticum, and from C. saccharolyticum, F. succinogenes, R. flavefaciens and Streptomyces sp., the β-glucanase gene from Trichoderma reesei and the avicelase gene from Thermatoga neapolitana have been isolated. Streptomyces viridosporus T7A oxidatively depolymerizes lignin as it degrades the cellulose and hemicellulose components of plant residues. The reactions produce a modified water-soluble, acid-precipitable polymeric lignin (APPL) as a major degradation product. Lignin peroxidase ALip-P3 enzyme encoded on a 4 kb fragment is one of four peroxidase-active proteins excreted by S. viridosporus (Wang et al., J. Biotechnol. 13: 131-144, 1990). ALip-P3 catalyzes C-C bond cleavage in the side chains of phenolic and nonphenolic lignin and oxidizes polymeric lignin. It is a heme protein with broad substrate specificity and oxidizes numerous substrates, including chlorinated aromatic compounds such as 2,4-dichlorophenol. The lignin depolymerizing enzyme system of S. viridosporus also includes extracellular aromatic acid esterases, aromatic aldehyde oxidases, and perhaps cellulases. The genes for liginin peroxidase from Phanerochaete chysosporium, lpo, was expressed in a baculovirus system. Genes from Phlebia radiata, lgp, Trameto versicolor and Bjerkandera adusta have also been identified. Thermomonospora mesophilia degrades lignocellulose and produces APPL. Streptomyces cyaneus, another lignin-solubilizing and APPL-producing actinomycete, grows on ball-milled straw and excretes an inducible extracellular protein involved in lignin solubilization. Streptomyces badius 252 excretes four extracellular peroxidases similar to those of S. viridosporus and produce one or more extracelluar oxidases which at least partially decompose lignocellulose.

### Effect of Chemicals on Soil and Groundwater

Herbicides used in agriculture also result in the contamination of groundwater and runoff and need to be monitored. The s-triazine ring is found as a constituent of herbicides, dyes, and polymers. The s-triazine herbicides including simazine, terbutylazine, and atrazine [2-chloro-4-(ethylamino)-6-(isopropylamino)-1,3,5-triazine)] are environmentally prevalent being used for the control of broadleaf and grassy weeds in major crops like corn, sorghum, and sugarcane. Residues of atrazine are found in ground, surface, drain and drinking water and seasonally exceed the safe concentration. The dechlorination of atrazine to yield hydroxyatrazine has been observed in plants like corn and animals. Hydroxyatrazine is thought to be nonherbicidal and nontoxic and does not leach from soil as readily as atrazine. Thus, hydrolytic dechlorination is ideal for metabolizing atrazine with the goal of environmental restoration. A Pseudomonas sp. strain ADP metabolized atrazine to carbon dioxide and ammonia via the intermediate hydoxyatrazine. A 1.9-kb avai DNA fragment from strain ADP contains the 1,419 nucleotide aztA gene encoding the atrazine-transforming activity, a 473-amino acid protein with a predicted molecular weight of 52,421 that has highest amino acid sequence identity with TrzA, a dechlorinating enzyme from R. corallinus NRRL B-15444R. AtzA is a chlorohydolase that catalyzes the dechlorination of atrazine, simazine, and desethylatrazine in soils and groundwater. AtzA confers atrazine dechlorination ability on Escherichia coli DH5α (De Souza et al., J. Bacteriol. 178: 4894-4900, 1996).

Organoclorine pesticides like DDT, Dieldrin and Lindane that have high lipoid solubility and are resistant to biodegradation can accumulate in animal tissues and produce long-term toxic effects. This results in enhanced formation and excretion of D-glucuronic acid and L-ascorbic acid, which further aggravates toxicity. (Street and Chadwick, Ann. NY Acad. Sci. 258: 132-143, 1975). Hepatic aldehyde reductase (AR1) reduces aliphatic and aromatic aldehydes, whereas carbonyl reductases CR1 and CR2 catalyze the reduction of aromatic aldehydes and ketones as well as quinones. Chlorodecone (Kepone), a toxic organochlorine pesticide, undergoes bioreduction to chlordecone alcohol in human liver by chlordecone reductase (CDR), of the aldo-keto reductase family of xenobiotic metabolizing enzymes. Three similar cDNA inserts coding for CDR were cloned and the protein had a molecular mass of 37.4 KD (Winters et al., Biochemistry 29: 1080-1087, 1990). An inducible carbonyl-reductase in gerbil liver catalyzes the bioreduction chlorodecone. Enzymes from white-rot fungi can degrade complex insoluble mixtures of pollutants like creosote and Arochlor. Enzymes from a Penicillium sp. are capable of methylating organic arsenic from pesticides and defoliants used in agriculture.

A Burkholdeia sp. strain PS12 degrades 1, 2, 4-trichlorobenzene and 1, 2, 4, 5-tetrachlorobenzene. A 5.5 kb DNA sequence from PS12 containing the tec genes coding for a chlorobenzene dioxygenase, a ferrodoxin and a reductase was expressed in E.coli (Beil et al., 1997, Eur J. Biochem. 247: 190-199). This resulted in the attack of aromatic compounds like chlorinated benzenes and toluenes, and biphenyl and dibenzo-p-dioxin.

Organophosphorus (OP) compounds like insecticides, fungicides and herbicides can accumulate in food products and water supplies. Microorganisms like Pseudomonas diminuta MG and Flavobacterium sp. ATCC 27551 possess high levels of organophosphorus hydrolase enzyme, which has a broad substrate specificity and catalyses the hydrolysis of phosphotriester bonds in OP pesticides such as methyl and ethyl parathion, paraoxon, dursban, coumaphos, cyanophos and diazinon as well as the phosphonate-fluride bonds of chemical warfare agents such as sarin and soman. A phosphotriesterase from P.diminuta that detoxifies OP pesticides has been isolated and the gene cloned (Serdar et al., Bio/Technol. 7: 1151-1155, 1989). The 1.3 kb fragment contains an ORF of 975 bases, coding for an enzyme of 35.4 KD that forms a dimer of 65 KD. OPH is a membrane-associated protein with an N-terminal signal sequence and recombinant OPH has been produced in sf9 insect cells, E.coli and secreted in soluble form by Streptomyces lividans. rOPH conferred paraoxon-resistance to fall armyworms and Drosophila melanogaster. It retains activity when anchored to outer surface of E.coli cell walls. The organophosphate paraoxon inhibits enzymes like cholinesterases and carboxylesterases in several tissues by binding to them. Carboxylesterases are important for the detoxification of drugs, pesticides, by preventing their interaction with acetylcholinesterase (Kaliste-Korhonen et al., Hum. Exp. Toxicol. 15: 972-978, 1996). A paraoxon hydrolyzing enzyme (Pxase) can detoxify in species-specific manner. This arylesterase/paraoxonase also hydrolyzes the insecticide clorpyrifos or Dursban. A parathion hydrolase gene specified by the Flavobacterium opd (OP-degrading) gene has been isolated (Mulbry et al., J. Bacteriol., 171: 6740-6746, 1989). The expression of a mouse opd gene, mpr56-1, has been recently detected in kidney and liver.

Nitriles are cyanide-substituted carboxylic acids used industrially in benzonitrile herbicides and as precursors for the synthesis of polyacrylonitrile plastics. They are also used as chemical solvents, extractants and recrystallizing agents in a number of industrial operations. They are released into the environment via industrial waste waters, and automobile exhaust gases which contain 1 μg hydrogen cyanide and 100 μg acetonitrile/ml. Most of them are highly toxic, mutagenic and carcinogenic. Pseudomonas marginalis is capable of metabolizing acetonitrile into ammonia and acetate (Babu et al., Appl Microbiol. Biotechnol. 43: 739-745, 1995). A two-step enzymatic mechanism—nitrile aminohydrolase, transforms the nitriles to their respective amides, and the amide is degraded by an amidase to its carboxylic acid and ammonia. Substrates for nitrile aminohydrolase are acetonitrile, phenyacetonitrile, isobutyronitrile, methacrylonitrile, butyronitrile, propionitrile and succinonitrile, whereas amidase exhibits maximum activity in the presence of acetamide, followed by propionamide, adipamide, benzamide, isobutyramide and methacrylamide. As these enzymes are able to produce carboxylic acids from nitrile compounds, they may be employed commercially in the production of the respective organic acids.

Industrially produced halogenated aromatic compounds constitutes a major class of environmental pollutants. In the case of the haloaromatics, the major degradative route involves conversion to corresponding halocatechols, intradiol (ortho) cleavage of the aromatic ring, and halide elimination during a subsequent reaction. Bacterial gene products are useful in the degradation of haloaromatics, toluenes, xylenes, and related aromatic hydrocarbons . The xylD and xylL genes of Pseudomonas sp. Strain B13 code for the enzymes toluate 1,2-dioxygenase and dihydro, dihydroxybenzoic acid dehydrogenase, while the nahG gene codes for salicylate hydroxylase. Combined expression of these genes from separate metabolic pathways led to the degradation of 4-chlorobenzoate, 3,5-dichlorobenzoate, salicylate and chlorosalicylates (Lehrbach et al., J. Bacteriol. 158: 1025-1032, 1984). Thus genetic engineering allows the combination of genes from different bacterial species and the use of essential DNA fragments avoids the introduction of unproductive enzymes. Nitroaromatics, such as nitrobenzenes, nitrotoluenes, nitrophenols, and nitrobenzoates, are of considerable industrial importance. They are frequently used as pesticides, explosives, dyes, polymers, pharmaceuticals or in the production of these compounds and serve as solvents or precursor for aminoaromatic derivatives. Several thousands of tons of these compounds (e.g. 2,3,6-trinitrotoluene and nitrobenzene) are produced annually. Many nitroaromatics have been shown to be toxic or mutagenic to many life forms. 2,4,6-Trinitrotoluene (TNT), 2,3-dinitrotoluene (2,4-DNT), and 1,3-dinitrobenzene, are toxic to many bacteria, yeasts, fungi, unicellular algae, todepool copepods and oyster larvae and they cause hepatitis and anemia in humans. Many organisms are able to reduce nitroaromatics (Marvin-Sikkema et al., Appl. Microbiol. Biotechnol. 42: 499-507, 1994). The degradation of nitroaromatic compounds occurs in situ, in soil, water, and sewage. Dense populations of nitroaromatic-degrading bacteria and nutrients, such as starchy waste are added to contaminated soils. Under these conditions, nitroaromatic herbicide dinoseb was degraded to non-toxic products such as acetic acid within 2 weeks. Comamonas acidovorans NBS-10 is also capable of producing oxygen-labile catechols from nitroaromatics.

Bacteria from the genus Rhodococcus contain a variety of enzymes that degrade halogenated hydrocarbons, numerous aromatic compounds. Thus they may be used to desulfurize compounds like coal or petroleum, or to accumulate cesium. Burning sulfur-containing petroleum and coal contributes to environmental degradation. Removal of inorganic sulfur from these fuels may be accomplished by physical, chemical, or biological means, but organically bound sulfur is difficult to remove. The gram-positive bacterium Rhodococcus sp. strain IGTS8 can extract sulfur from a variety of organosulfur compounds, petroleum and soluble coal derived materials, including thiophenes, sulfides, mercaptans, sulfoxides, and sulfones by breaking carbon-sulfur bonds, releasing sulfur in a water-soluble, inorganic form. Rhodococcus sp. strain IGTS8 possesses an enzymatic pathway that can remove covalently bound sulfur from dibenzothiophene (DBT) without breaking carbon-carbon bonds. The products of three genes designated soxABC (sulfur oxidation), expressed as an operon, were required for DBT desulfurization to 2-hydroxybiphenyl (Denome et al., J Bacteriol. 176: 6707-6716, 1994). The soxABC genes conferred the DBT desulfurization phenotype to desulfurization-negative mutants of IGTS8 and to another species, Rhodococcus fascians. Thus, with the appropriate regulatory signals the enzymes could be active in a genera beyond rhodococci.

Among the other biotransformations that rhodococci catalyze are steroid modification and transformation of nitrites to amides and acids. Some strains produce enzymes like phenylalanine dehydrogenase and endoglycosidases. A non-heme haloperoxidase is involved in the biodegradation of thiocarbamate herbicides by Rhodococcus erythropolis NI86/21 and a 30 Kda protein is encoded by the thcF gene (De Schrijver et al., Appl. Environ. Microbiol. 63: 1911-1916, 1997). This is homologous to the gene for a chloroperoxidase from Pseudomonas pyrrocinia.

2-Hydroxybiphenyl has been used as a fungicide of various fruits since 1937. 2-hydroxy- and 2,2′-dihydroxybiphenyl are also the end products of the bacterial desulfurization of dibenzothiophene, a major sulfur-containing component of fossil fuels. In rats, 2-hydroxybiphenyl shows renal toxicity and causes tumors of the urinary bladder. 2-Hydroxybiphenyl 3-monooxygenase, an aromatic hydroxylase encoded by the hbpA gene of Pseudomonas azelaica HBP1, catalyzes its conversion to 2,3-dihydroxybiphenyl (Suske et al., J. Biol Chem. 272: 24257-24265, 1997). It has sequence homology to 2,4-dichlorophenol 6-hydroxylase from R. eutropha and phenol 2-hydroxylase from Pseudomonas sp. strain EST1001.

Styrene is a toxic compound used in large amounts by the chemical industry and released into the environment. Styrene contamination can occur by factory waste water, evaporation and the pyrolysis of polystyrene. A 4,377-bp chromosomal region of Pseudomonas fluorescens ST contains the styA and styB genes encoding a styrene monooxygenase responsible for the transformation of styrene to epoxystyrene, and styc encoding an epoxystyrene isomerase which converts epoxystyrene to phenylacetaldehyde, which is subsequently oxidized to phenylacetic acid by a styD-encoded phenylacetaldehyde dehydrogenase (Beltrametti et al., Appl. Environ. Microbiol. 63: 2232-2239, 1997).

### Urinary Tract Structure and Function

The human kidney is comprised of approximately 1,000,000 nephrons, as described in Best and Taylor's Physiological Basis of Medical Practice, 11th Ed., J. B. West; Physiology, 2dn Ed., Berne and Levy, CV. Mosby Co., 1988. See FIGS. 1 and 2.

The kidney regulates the composition of the extracellular fluid by selectively adjusting the composition of the plasma that flows through the renal vasculature and providing a relatively constant environment for the normal functions of the cells. The kidney also plays a role in the production of hormones such as angiotensin II, prostaglandins, and the kinins, all of which are involved in the regulation of blood pressure. The kidney also monitors the adequacy of oxygen delivery to the tissues and synthesizes erythropoietin, a glycoprotein hormone that regulates the production of red blood cells from precursor cells in the bone marrow in response to renal hypoxia.

The formation of urine involves three processes: filtration, reabsorption, and secretion. Materials to be conserved are retained in the plasma, and waste products are extracted and excreted. End products of hepatic metabolism frequently appear in the urine in the form of organic anions. For example, uric acid, the end product of purine metabolism is eliminated exclusively by the kidneys. Table I, below, lists organic materials secreted by the proximal tubule of the kidney.

TABLE I

| | |
|---|---|
| Endogenous Substances | Drugs and other Exogenous substances |

(A) Organic acids secreted by the proximal tubule

| | |
|---|---|
| Bile acids | Cephalothin |
| cAMP | Chlorothiazide |
| Hydroxy indoleacetic | Ethacrynic acid |
| acid | |
| Oxalic acid | Furosemide |
| Uric acid | Iodohippuric acid |
| | p-Amino hippuric acid (PAH) |
| | Penicillin |
| | Salicylic acid |

(B) Organic bases secreted by the proximal tubule

| | |
|---|---|
| Acetylcholine | Amiloride |
| Creatinine | Atropine |
| Dopamine | Cimetidine |
| Epinephrine | Hexamethonium |
| Histamine | Isoproterenol |
| Norepinephrine | Morphine |
| Serotonin | Neostigmine |
| Thiamine | Procaine |
| | Quinine |
| | Tetraethylammonium |
| | Trimethoprim |

Urea, the major end product of protein metabolism is eliminated exclusively by the kidneys. Additionally, proximal tubular cells synthesize ammonia.

The kidneys of mammals differ morophologically from those of amphibinans and reptiles in two predominant respects. First, the nephrons have loops of Henle interposed between their proximal and distal convoluted segments, and second, the loops of Henle and collecting ducts are organized into parallel arrays. Birds share these features to a degree. In fish, the renal organs are similar to that seen in amphibians. In both birds and reptiles, the ureters discharge into the cloaca. Principal nitrogenous end products may be different in certain groups of animals, and genes may be added to various species to alter the end products of metabolism of nitrogen compounds. (Animal Physiology, Eds. Hill R W and Wyse G A, 1989; Comparative Vertebrate Anatomy, Ed. Hyman H L, 1978; The Physiology of Fishes, Evans D H, 1997; Wright et al., J. Exp. Biol. 198: 273-281 (1995).

Thus, the kidney is a complex organ and urine is a mixture of water, ions and proteins, some of which are potential sources of pollution when found in large quantities in the environment.

### SUMMARY OF THE INVENTION

Thus, a need exists for controlling organic wastes associated with agriculture and animal husbandry. A need also exists for a method of altering urine so as to reduce its toxic effect. The present invention is based upon the discovery that transgenic animal techniques can be used to satisfy these needs. Specifically, one embodiment of the present invention relates to a method of producing a protein that degrades or detoxifies organic material. This method involves providing a non-human transgenic animal having stably integrated into its genome an exogenous gene encoding a protein that is detectable in urine and that degrades or detoxifies organic material. The animal used is a mammal selected from the group consisting of a pig, sheep, goat, cattle, rodent, rabbit, horse, dog, cat, but can also include non-mammals, such as a bird, fish or reptile. The protein encoded can be an enzyme, such as the enzymes listed in FIG. 7. The organic material to be degraded or detoxified is feces, guano, urine, a microbe, chemical pollutant and a by-product thereof or a food product and by-product thereof. Specifically the chemical pollutant could be a herbicide, a pesticide, including an insecticide, or a fertilizer.

In another embodiment, the invention relates to a method of degrading or detoxifying organic material, comprising the steps of providing a non-human transgenic animal that produces in its urine a protein that degrades or detoxifies an organic material, where the non-human transgenic animal has stably integrated into its genome an exogenous gene encoding such protein that is detectable in urine. The method comprises the steps of: (a) providing a non-human transgenic animal having stably integrated into its genome an exogenous gene encoding a protein that is detectable in urine and that degrades or detoxifies organic material; and (b) contacting the organic material with the urine, thereby degrading and detoxifying the organic material. The contacting may involve mixing the urine with the organic material or having the non-human transgenic animal urinate on the organic waste.

In yet another embodiment, the invention relates to a facility for containing animals. This facility comprises at least one non-human transgenic animal having stably integrated into its genome an exogenous gene encoding a protein that is detectable in urine and that degrades or detoxifies organic material; and a structure for containing the animal within the facility. The facility also includes at least one non-transgenic animal of the same or different species from the transgenic animal. The transgenic animals are described as above. Both the transgenic animal and the non-transgenic animal may be mammals or in another embodiment, the non-transgenic animal may be a bird or reptile. In yet another embodiment, the transgenic animal may be a bird, such as a chicken, turkey, goose or duck. The facility is a farm, ranch, slaughter house, research facility or zoo. The structure could be a building, cage, fence or other enclosure typical for containing animals.

In another embodiment, the present invention relates to an a method of altering the natural composition of urine. More specifically, it relates to an in vivo method of altering a substance in urine, the method includes producing a non-human transgenic animal that has stably integrated into its genome an exogenous gene encoding a first substance that alters a second substance in the urine of the transgenic animal. The first substance may degrade the second substance. The first substance may be a protein, and preferably it is an enzyme as described in FIG. 7.

In yet another embodiment, the invention relates to a gene construct for use in transgenic animals. This construct comprises (a) 5′ expression regulating sequences, including urinary tract-specific promoter and enhancer sequences; (b) cDNA or genomic DNA sequences encoding complex peptides and proteins with enzymatic activity, and a signal sequence effective in directing the secretion of said peptide or protein into the urine of transgenic animal; and (c) 3′ regulatory sequences, including a polyadenylation sequence, that results in the expression of said DNA sequences in the urinary tract cells; wherein a, b and c are operably linked in said gene construct to obtain the production of said peptide or protein in urinary tract cells and secretion into urine of an animal.

In an additional embodiment, this invention provides a non-human male or female transgenic animal comprising cells having incorporated expressibly therein a polynucleotide encoding a complex protein or peptide that is produced in the urine. This gives an unique opportunity to utilize the urinary tract as a site for production of recombinant proteins. The transgenic animals produce complex heterologous proteins, enzymes or peptides in their urine, wherein the composition of the urine is altered or the components of urine are modified.

Further, the complex proteins, enzymes or peptides are produced in the urinary tract of the animal and are present in the urine of the animal. The regions of urinary tract include the kidneys, the ureters, the bladder and the urethra. The cells of the kidneys and bladder are the epithelial cells, and the preferred regions for expression are the distal tubules of the kidney or the bladder.

The cDNA or genomic sequences encoding the protein of interest, along with signal sequences for secretion may be used, as also entire gene loci or operons. Minigenes containing homologous or heterologous introns may also be used.

### A BRIEF DESCRIPTION OF FIGURES

FIG. 1 depicts a longitudinal section of the kidney.

FIG. 2 depicts the structure of the nephrons of the kidney.

FIG. 3A depicts a WAP/HPC construct.

FIG. 3B represents human Protein C structure and function. Specifically, the 461 amino acid precursor with cleavage sites is presented. The arrows indicate protein cleavage sites, the numbers denote amino acid residues. Gla: γ-carboxyglutamic acid, EGF: epidermal growth factor-like domain, OH: β-hydroxyaspartate, CHO-oligosaccharides, AP: activation peptide, Ser, His, Asp: residues of the catalytic triad, PL: phospholipid, T/TM: thrombin/thrombomodulin, PF4: platelet factor 4, α₂-MAC: α₂-macroglobulin, PAI: plasminogen activator inhibitor.

FIG. 4 shows a Northern blot analysis of total RNA (1, 3, 5, 7) and mRNA (2, 4, 6, 8) from tissues of mice transgenic for HPC. Transcripts from human liver (lanes 1-2), the mammary gland (lanes 3-4) and kidney (lanes 5-6) of WAP/HPC transgenic mouse 4.2.10.9 (Drohan et al., Transgenic Res., 3: 355-364 (1994) and human liver HepG2 cells (lanes 7-8) were analyzed. To obtain signals of similar intensity, different amounts of RNA were loaded in lanes 1 through 8; 3.7, 0.11, 0.004, 0.0001, 3.7, 0.096, 2.1 and 0.021 μgs respectively. Blots were hybridized with HPC cDNA probes as in (Drohan et al., 1994, supra). The arrow indicates the mature rHPC transcript, RNA standards in kilobases are given on the left.

FIG. 5 shows a Western Blot Analysis that was carried out to detect rHPC in protein fractions enriched from the urine of a transgenic pig, after 10% SDS-polyacrylamide gel electrophoresis. (CON): urinary proteins in dialyzed urine from a control pig, (TRG): urinary rHPC eluted after chromatography from urine of a WAP/HPC transgenic pig, (HPC): plasma-derived HPC standard. HC: heavy chain and LC: light chain of HPC; kDa: molecular weight in kilodaltons.

FIG. 6 depicts general gene constructs for expression in the urinary tract.

FIG. 7 lists enzymes suitable for use in degrading or detoxifying organic materials.

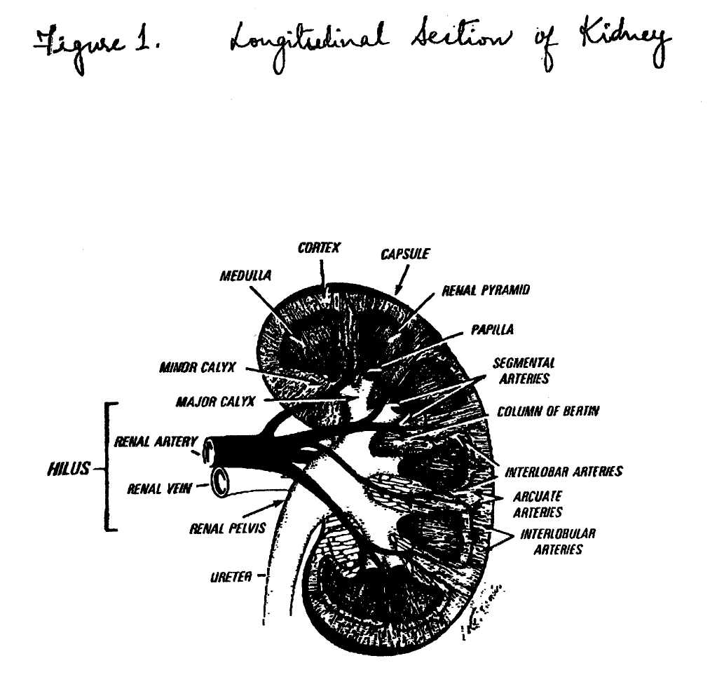

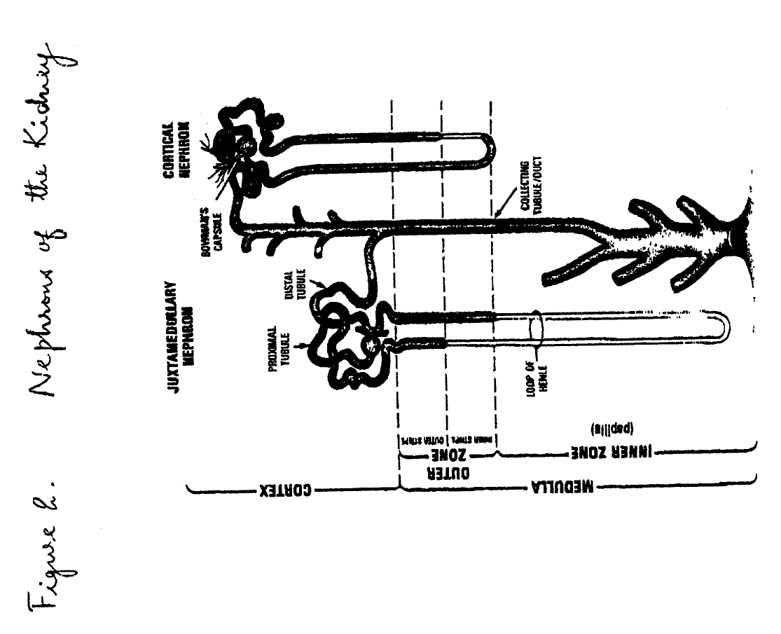

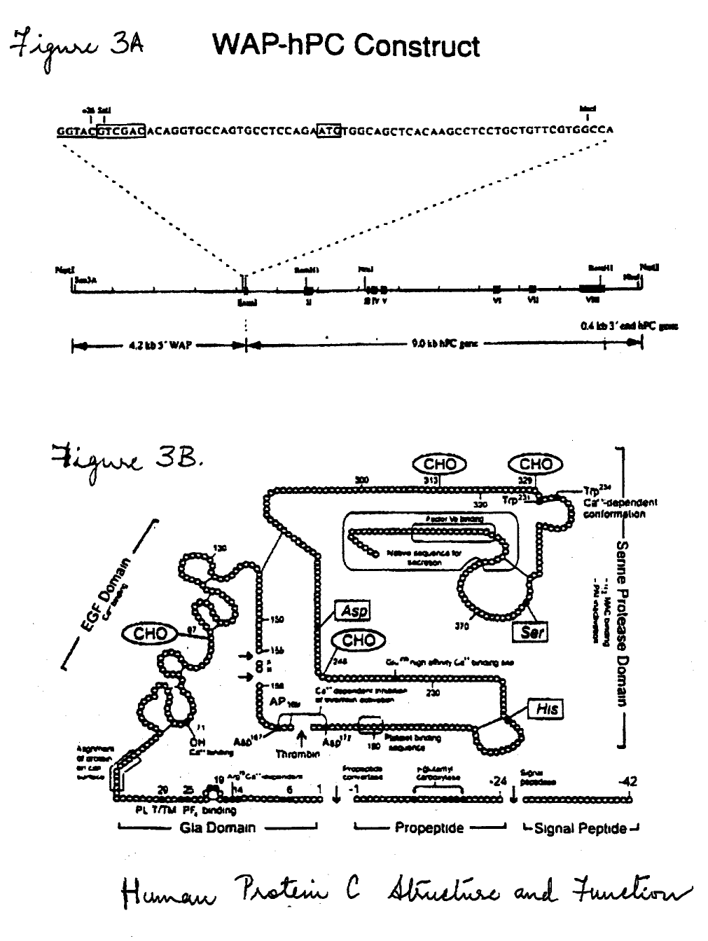

### DETAILED DESCRIPTION OF THE INVENTION

In one embodiment, the invention relates to a method of producing a protein that degrades or detoxifies organic material. In another embodiment, the invention relates to a method of degrading organic material. Both embodiments require a non-human transgenic animal that produces in its urine a protein that degrades or detoxifies organic material. The non-human transgenic animal has stably integrated into its genome an exogenous gene encoding a protein that is detectable in urine. The protein is detected using protein detection methods well known to persons skilled in the art, such as immunological or enzymatic assays, Western blots, and other known methods. By production of the protein in the urine is meant the expression of the exogenous gene in cells in the urinary tract, which results in the protein being ultimately detectable in the urine. In a preferred embodiment the animal is a mammal, such as a pig, sheep, goat, cattle, rodent, rabbit, horse, dog and cat. In the most preferred embodiment the protein is an enzyme. In another preferred embodiment the animal is a bird, such as a chicken, hen, duck, goose or turkey, or is a fish or reptile. Organic materials that are degraded or detoxified by such enzyme include organic material in feces, guano, and urine, as well as chemical polluntants or by-products thereof, such as fertilizers, herbicides (including fungicides), pesticides, insecticides, microbes, food products and food by-products, such as cellulose.

In some cases, the organic materials degraded or detoxified by the protein of the invention is produced by the non-human transgenic animal itself. In other cases, the organic material is produced by non-transgenic animals of the same or different species from the transgenic animal. For instance, the transgenic animal is a mammal, such as a pig and the organic material is produced by birds or reptiles or other wild animals, in the case of a poultry farm or zoo, respectively. In yet other cases, the organic material is food or a food by product associated with agriculture or the care of animals. As such, this method of the present invention can be used on farms, slaughter houses, ranches, zoos, research institutions or any other type of facility where animals are contained or plants are grown. The urine produced by the non-human transgenic animal degrades and detoxifies the urine or feces produced by itself and/or other animals in such facility. Urine alone or mixed with feces and other wastes produced by the transgenic animal may be collected for purposes of distributing on or mixing with organic materials or it may come in contact with organic materials by the transgenic animal directly urinating on the organic material.

By “degrading” is meant the complete or partial breaking down of the natural structure of a protein or other organic compound. By “detoxifying” is meant rendering the protein or other organic compound non-toxic. Detoxification may occur due to a complete degradation or a modification in the protein or its function.

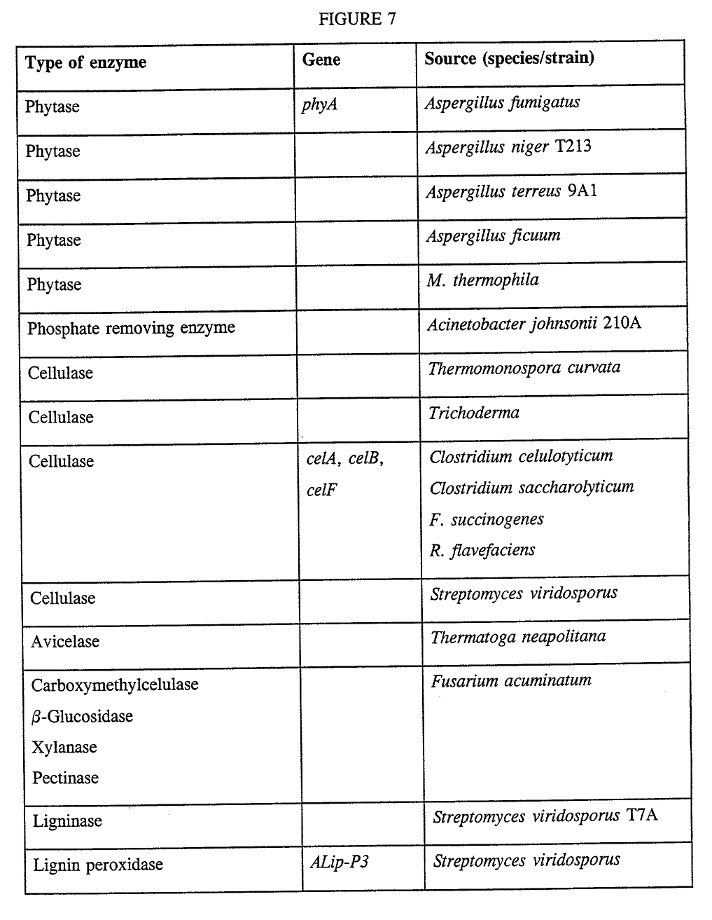

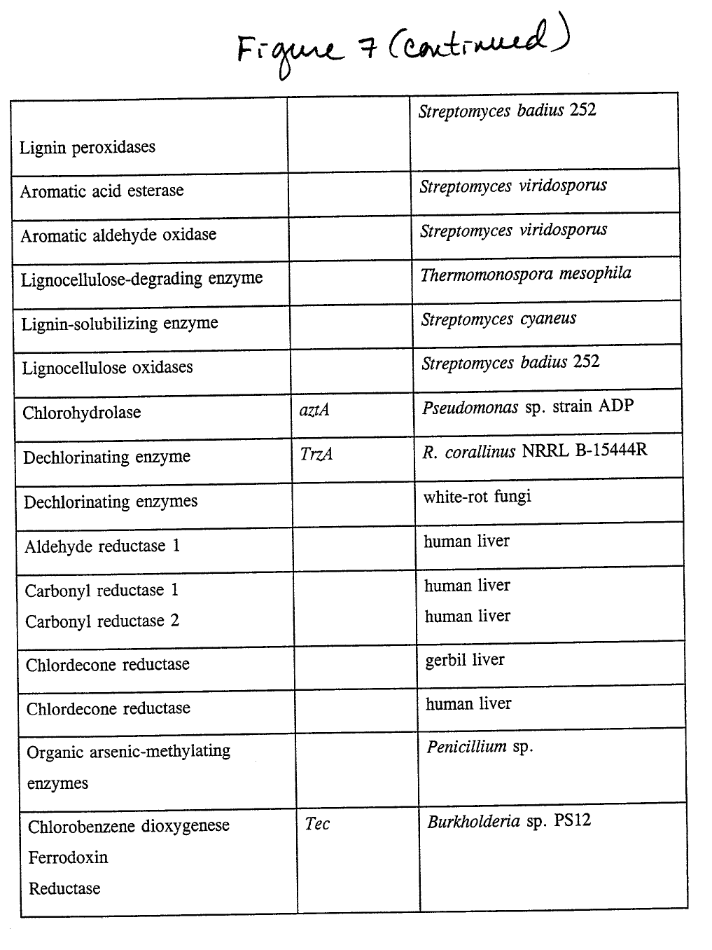

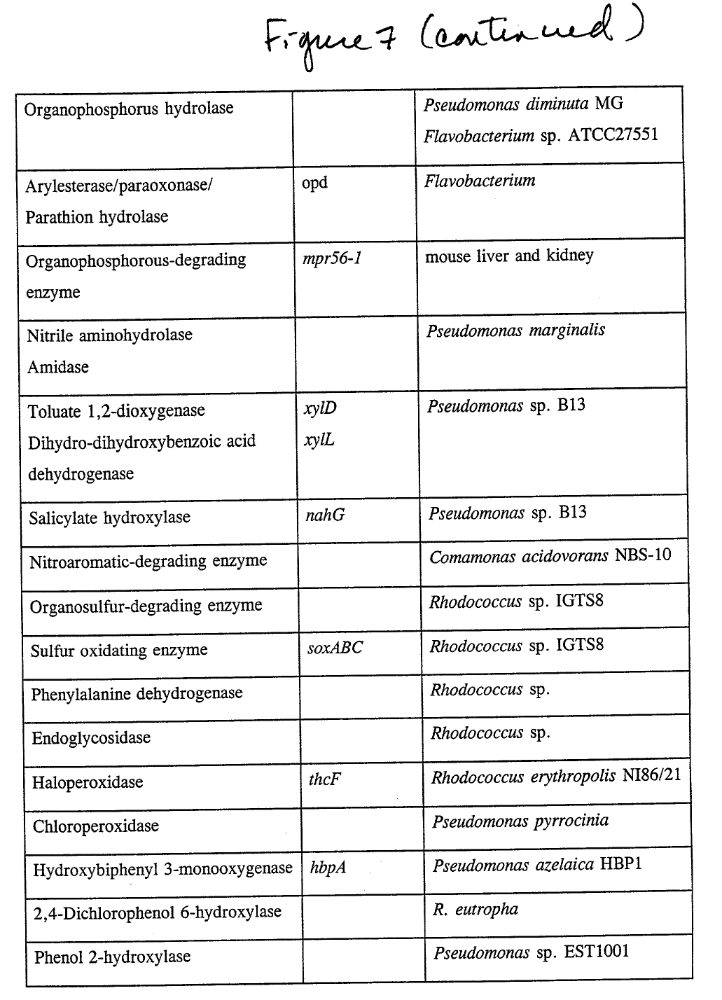

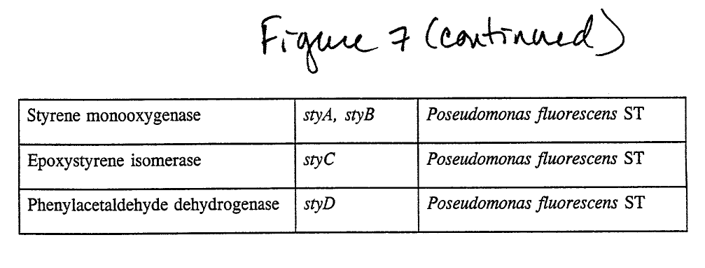

By “protein” is intended peptides and fragments of proteins, as well as mutants and variants of proteins. Specific proteins according to this invention include all enzymatically active proteins or peptides from bacterial, fungal or plant sources that affect the composition of animal waste. Very useful are enzymes that are active at very high or very low temperatures. Particularly useful proteins are enzymes from microorganisms that are pre-adapted to convert waste products. For example, enzymes exist that degrade penicillin antibiotics in excreta and reduce the formation of antibiotic-resistant bacteria and their spread in the environment. Examples are biologically active peptides or proteins that affect the composition of the soil and/or surface and groundwater. Other enzymes detoxify pesticides that contaminate soil and water. The identification of additional proteins and genes that express such proteins is within the skill of the art using techniques that are well known. Methods for manipulating known proteins and related DNA from bacterial, viral, fungal, plant and animal genomes are similarly well-known. Thus, the proteins of the present invention include all known and possible variants or modifications of proteins that detoxify or degrade organic materials. The invention also relates to proteins that are expressed in a transgenic animal and then may or may not be posttranslationally modified by a different protein. Examples of enzymes according to the present invention are set forth in FIG. 7.

In another embodiment, the present invention relates to a transgenic animal that produces in its urine a protein that degrades or detoxifies organic material. The production of such transgenic animal requires the application of skills well known and accepted in the art. For instance, one can obtain DNAs for producing transgenic organisms by applying conventional methods of recombinant DNA cloning. A general discussion of well known techniques for making suitable DNAs in this regard is provided by Maniatis et al., Molecular Cloning, A Laboratory Manual (Cold Spring Harbor Laboratory, 1982) and Sambrook et al., Molecular Cloning, A Laboratory Manual, Second Edition, Vol. 1-3 (Cold Spring Harbor Laboratory, 1989), which are incorporated by reference herein, in pertinent part. Examples of DNA constructs that have been introduced into transgenic animals for systemic or tissue-specific expression are provided in GENETIC ENGINEERING OF ANIMALS, A. Puhler, Ed., VCH Verlagsgesellschaft, Weinheim, N.Y. (1993), which also is incorporated by reference herein in pertinent part in this regard.

DNA coding for a given protein can be fused, in proper reading frame, with appropriate regulatory signals, as described in greater detail below, to produce a genetic construct which then may be amplified, for example, by propagation in a bacterial vector or by PCR, for subsequent introduction into a host organism, according to conventional practice. Generally, the genes will be linked operatively to the cis-acting signals necessary for expression in a desired manner in an organism. Particularly preferred in this regard are promoters and other cis-acting regulatory elements that provide efficient expression in a particular cell-type. In the following discussion, the term promoter is used broadly and extends to cis-acting elements such as enhancers that may not always be considered in a strict technical sense, promoters.

The cis-acting regulatory regions useful in the invention include the promoter used to derive expression of the gene. Particularly useful in the invention are those promoters that are active specifically in given cell-types. In this regard, preferred promoters are active specifically in cells that secrete substances into bodily fluids such as urine. Notably, therefore, cells of urinary tract are especially useful. Most useful are tubular epithelial cells of the kidney and epithelial cells of bladder. Particularly preferred promoters in this regard are those which are active in urinary tract tissue, such as kidney or bladder tissue. Most preferred in this regard are those that are both specific to and efficient in urinary tract tissue. By “efficient” is meant that the promoters support the synthesis of reasonably large amounts of protein in urine.

It is within the skill of the art to isolate such promoters and other regulatory sequences from proteins associated with urinary tract tissue. For instance, uromodulin or the Tamm-Horsfall protein (THP) is found in human urine in large quantities, 20 to 200 mg per day, or 15-37% of total protein. THP expression is specific only to the kidney and not to liver, heart, lung, brain, thymus, muscle, spleen, testis or placenta. The single human gene consists of 11 exons and 10 introns, the mRNA is about 2.6 kb in size (Hession et al., Science 237: 1479-1484, 1987; Pennica et al., Science 236:83-87, 1987). Nucleotide sequencing of a full-length cDNA predicted a protein of 640 amino acids, that includes a 24 amino acid leader sequence and a mature protein with 616 amino acids including 48 cysteine residues. The leader sequence suggests that the majority of THP is a secreted protein, but is a glycosylphosphatidyl inositol-linked membrane protein that is released by the action of a phospholipase or a protease, to form large aggregates in urine. The mouse uromodulin cDNA predicts a 642 amino acid protein. THP was initially isolated from the urine of pregnant women as a protein having the ability to inhibit antigen-induced T-cell proliferation. It is also present in urine from males. THP has 8 potential glycosylation sites, of which only five are utilized. The carbohydrate moiety of uromodulin is a specific ligand for cytokines, such as interleukin-1, interleukin-2 and tumor necrosis factor. The ability to bind type 1 pili of Escherichia coli, possibly allowed it to provide protection from bacterial colonization of the urinary tract. Human and sheep THP interact with high affinity with human and sheep IgG. Four domains of THP exhibit similarity to the cysteine-rich region of the epidermal growth factor precursor and the low density liporotein receptor. Two asparagines are hydroxylated. THP also expresses an “RGD” sequence as in fibronectin, fibrinogen, type 1 collagen, and thrombospondin, which bind to cell surface receptors. It may also play a role in the maintenace of water and salt balance in the loop of Henle.

THP was consistently found in the 16th week of gestation and was detectable after the 20th week in amniotic fluid, rising to a median value of 1.3 mg1⁻¹ at birth. Postnatally, THP excretion increases steadily, reaching a maximum in early adulthood. It localized to the ascending limbs of Henle's loop and early distal tubule, on both apical and basolateral surfaces. THP-excretion is low immediately after renal transplantation and increases to normal values 2-3 weeks later. THP of diabetics had significantly different sugar content and altered colloid stability, with no significant differences of amino acids. Cisplatin causes profound proximal tubular damage and no distal tubular cell injury, with a temporary rise in THP excretion due to increased diuresis and increased distal tubular urine flow due to reduced proximal tubular fluid reabsorption.

The luminal surface of mammalian urothelium is covered with numerous plaques composed of semi-crystalline, hexagonal arrays of 12-nm protein particles involved in stabilizing the urothelial surface during bladder distention. A 27-kD urothelial plaque-associated (uroplakin I) protein is expressed in superficial umbrella cells during differentiation. A 15-kD urothelium-specific protein uroplakin II and III were also identified. The uPA II cDNA encodes a protein that is anchored in the membrane by its C-terminal region, with the N-terminal domain exposed to the lumen (Lin et al., J. Biol. Chem. 269: 1775-1784, 1994). UPA gene expression is bladder-specific and differentiation-dependent. A 3.6 kb 5′ flanking sequence of the uPAII gene could target expression of a bacterial lacZ gene to the differentiated suprabasal cells of the urothelium (Lin et al., Proc. Natl. Acad. Sci. 92: 679-683, 1995).

Erythropoietin (EPO) is a glycoprotein that promotes the proliferation and differentiation of erythrocyte precursors. The major site of EPO production is the kidney cortex and to lesser extent the outer medulla, while the liver is the main extrarenal site. EPO production in response to acute hypoxia represents de novo synthesis and is regulated by changes in EPO mRNA. EPO mRNA was found in the tubular fraction but not in glomerular tissue. EPO-producing cells in the kidney were peritubular cells, mainly the endothelial cells (Lacombe et al, J. Clin. Invest. 81: 620, 1988).

Soluble human Thrombomodulin (TM) is present in both plasma and urine of normal subjects (Yamamoto et al. J. Biochem. 113: 433-440 (1993)). The urinary thrombomodulins may have been produced by cleavage of cellular thrombomodulin by elastase or elastase-like enzymes, but it is not probable that plasma TM is physiologically filtrated into urine through the glomerular membrane, as the molecular weight of soluble TM in plasma is 28-105 kDa. They may be produced by kidney cells. Two major molecular forms of thrombomodulin fragments present in urine were isolated from human urine by four sequential steps of column chromatography. UTM lacked 29 amino acids of the carboxyl-terminal sequence of intact cellular thrombomodulin and Type II had less galactosamine than type I. The urinary thrombomodulins contained the N-terminal EGF-like domain essential for thrombin binding and protein C activation, and the initial 6 residues of the putative O-glycosylation-rich domain. Three potential O-glycosylation sites in the O-glycosylation-rich domain are missing.

Urine also contains nephrocalcin (NC), a calcium oxalate monohydrate crystal growth inhibitor. Primary mouse proximal tubule cell cultures produce NC. Purification showed that all NC are glycoproteins with 10-20 wt % of carbohydrate. They had a high content of acidic amino acid residues (aspartic acid and glutamic acid) but few aromatic and basic amino acid residues. All NCs contain fucose, galactose, glucose, mannose, galactosamine, glucosamine, and traces of N-acetylneuraminic acid. The elevation of urinary NC in patients with renal cell carcinoma is common and results from tumor growth rather than a biochemical alteration in normal kidney cells. Urinary levels of NC corresponded with disease progression in patients with metastatic disease. An osteocalcin-related gene has been identified as the nephrocalcin gene in mice (Desbois et al., J. Biol. Chem. 269: 1183-1190, 1994) and is transcribed only in kidney, not bone.

A urinary stone inhibitor protein detected in the cells of the descending limb of the loop of Henle and in papillary surface epithelium at the calyceal fornix, where urine is highly concentrated in stone mineral constituents, was found to be identical to be osteopontin (OPN). OPN mRNA is found at high levels in the kidney, the protein is synthesized and secreted into tubule fluid by the epithelium in the thick ascending loop of Henle and the distal convoluted tubules. Female, pregnant and lactating mice expressed more OPN than males. As animals age, expression is found in more proximal portions. The characterization of osteopontin-k cDNA from bovine renal library showed that it was a kidney cell adhesion molecule of about 261 amino acids and 29.6 Kda molecular weight (Crivello et al., J. Bone Miner. Res. 7: 693-699, 1992).

The collecting duct apical membrane water channel (AQP-CD) of rat kidney is important for the formation of concentrated urine and its RNA is detected only in kidney. The cDNA of human aquaporin of the collecting duct (HAQP-CD or AQP2) encodes a 271-amino acid protein with 91% identity to rat AQP-CD. mRNA expression of hAQP-CD was predominant in the kidney medulla compared to the cortex, immunohistochemical staining of hAQP-CD was observed only in the apical domain of the collecting duct cells. rAQP-CD is the vasopressin-regulated water channel of the kidney collecting duct. It contains a consensus sequence for phosphorylation by protein kinase A in the C-terminal which is conserved in hAQP-CD. HAQP may be composed of two molecular mass forms of 29 kD and ˜40 kD. The 29-kD intact HAQP present in the urine of a normal subject indicates that HAQP-CD becomes detached from the membrane and is excreted into the urine. Aquaporin 3 (AQP3) is another water channel present in the epithelial cells of the rat medullary collecting duct and is encoded by a 1.9 kb cDNA.

The cDNAs for rat and rabbit vasopressin-regulated renal urea transporter which are involved in urea accumulation in the renal medulla were cloned. RUT2 was found in apical and subapical regions of inner medullary collecting duct and in terminal portions of the descending thin limbs. Rat UT1 expression was found in inner medulla and differed from rUT2.

Sex-related differences have been observed in protein composition of urine, with urine from normal human males containing much more Protein 1 (P1) than from non-pregnant females after puberty. Levels of UP1 have been measured with a latex immunoassay or ELISA. However, there was no sex difference in serum UP1 level. This is due to secretion of plasma P1 stored in the bladder after filtration from kidney and genital tissues. Other genes like that of transforming growth factor β, retinoid X receptors, erythropoietin receptor and dopamine 1A receptor are expressed in the kidney. Various urinary proteinases have been isolated from both normal urines and from urines from patients (Chawla et al., J. Cell Biochem. 50: 227-236 (1992).

Several serum proteins are found in urine of patients with various diseases. Urinary β2-microglobulin in end-stage renal disease consists of 2 of the 5 pI forms of plasma β2-M. Low molecular weight proteins like urine Protein 1 have been isolated from urine of patients with chronic renal failure (Itoh et al., J. Clin. Lab. Anal., 7: 394-400, 1993), and is highly elevated in diabetic and cadmium nephropathy. Patients with renal failure usually excrete large amounts of several plasma-derived urinary proteins, including albumin, α₁m, β₂m and retinol binding protein (RBP). Such urine also contained a large amount of P1. The presence in the urine of proteins like intestinal alkaline phosphatase (IAP), a marker of the pars recta (S3-segment) and villi, a proximal tubule-localized cytoskeletal protein which signifies brush border loss can be used as cellular markers of proximal tubular cell injury.

TABLE II
Concentration of Some Proteins in Urine

Concentration in Urine
(Range), μg/L

| Protein | General | Males | Females |
|---|---|---|---|
| Nephrocalcin | | 14.0 + 2.8 | |
| Protein 1 | | 14.2 (1.7- | 1.0 (0.2- |
| | | 42.7) | 4.2) |
| sCD58 | | 6.8 (4.8-8.8) | — |
| Thrombomodul | 102 ± 38 | — | — |
| in | | | |
| Trypsin | 5.71 (human), | — | — |
| Inhibitor | 5.0 (horse) | | |
| | mg/L | | |
| Uromodulin | 20-200 mg/L | — | — |

Total protein content urine = 7.4 × 10⁴ OD units/L or 74 g/L − 10.8 g/L

The purification of several proteins from the urine of humans and animals of various species has been reported. Ultrafiltration devices allow rapid recovery of milligram amounts of low molecular weight urinary proteins in concentrated form. Exhaustive dialysis provides a purer preparation of α2-μglobulin (Marshall et al., Biochem. Soc. Trans. 20: 1885 (1992)). Uromodulin was isolated by salt-precipitation (Tamm and Horsfall, J. Exp. Med. 95: 71-, 1952) and lectin adherence (Hession et al., Science 237: 1479-1484, 1987). Protein 1 was isolated by ammonium sulphate precipitation, immunoaffinity chromatography, gel filtration and ion exchange, rp-HPLC. Uropepsinogen was highly purified (Minamiura et al., J. Biochem. 96: 1061-1069, 1984), as were rat urinary kallikrein and normally excreted serum proteins in native form, like urinary acid-resistant trypsin inhibitor and soluble form of serum CD58 (LFA-3) from human and animal urine. Some proteins are more stable than others in urine, for example, P1 is very stable for at least 4 days, while β2 M is unstable beyond 24 hrs at 37 C. Peptide mapping has allowed the identification of angiotensin, urodilatin, psoriasin and granulin from urine.

Gene transfer into and expression in the kidney can be achieved in several ways. Embryonic kidney tissue can develop and differentiate when transplanted into the parenchyma of mouse kidneys in the postnatal period, allowing the transfer of novel genes into the mammalian kidney in vivo (Woolf et al., Exp. Nephrol. 1: 41-48, 1993). The implant develops to form vascularized, filtering glomeruli connected to differentiated renal tubules with open lumina. Tissue infected ex vivo with a replication defective retrovirus transduces the gene for β-galactosidase with gene expression predominantly in glomerular epithelial cells, but also in interstitial cells and vascular structures. Positive tubular cells were not found. In the rat, tubular expression does occur after metanephric transduction. This may be a species difference, or the earlier developmental stage at which the rat metanephros was transduced. Even nonfiltering nephrons might deliver gene products into the kidney by virtue of diffusion of these proteins. In contrast, the adult mammalian kidney has a low cell turnover with a mitotic index of less than 15 per 10⁵ cells, and is an unsuitable target for infection by retroviruses. Successful in vivo gene transduction into the adult rat kidney may be possible after the induction of tubular cell replication by a chemical nephrotoxin, or by using rapidly dividing embryonic kidney tissue as a cellular vector for the novel gene.

Transgenic animals may be generated using the promoters and other regulatory sequences of kidney- or bladder-specific genes, or by using the urinary tract-specific regulatory elements present in other genes, such as in the human apolipoprotein (apo) E gene. Constructs with 30 or 5 kb of 5′-flanking and 1.5 kb of 3′-flanking DNA were used to create transgenic mice and high levels of human apoE mRNA were produced in the kidney (Simonet et al., J. Biol. Chem. 265: 10809-10812, 1990). The source of human apoE in the transgenic kidney was the epithelial cells lining the proximal tubule and Bowman's capsule. The use of 23 kb of downstream regulatory elements, however, suppressed expression in the kidney. Rat apoE synthesis in the kidney was also limited to the epithelial cells of the proximal convoluted tubule. 6.5 kb of 5′ flanking sequence of the mouse EPO gene, along with 1.2 kb 3′ flanking sequences could target low level expression of the lacZ gene specifically to renal proximal convoluted tubule cells, which was increased by hypoxia induction. Regulatory sequences required for induction of hEPO in the kidneys of transgenic mice lie more than 9.5 kb 5′ of the human EPO gene. Sequences in other genes such as the milk protein gene, WAP, are also normally expressed at low levels in the kidneys of female virgin mice and during lactation (Wen et al., Mol. Reprod. Dev. 41: 399-406, 1995), although the presence of WAP protein has not been demonstrated.

Thus, the kidney is a viable organ for gene transfer and promoters suitable for use in preparing transgenic animals according the present invention include the promoters associated with the above described proteins, particularly the uromodulin and uroplakin promoters. It is within the skill of the art to isolate other promoters that are suitable in the present invention. Additionally, regulatory sequences of other genes may be modified to obtain kidney-specific expression. Preferred are the urinary tract-specific regulatory elements found in the 5′ and 3′ regulatory sequences of the human apolipoprotein E gene or the mouse renin, Ren-2 gene, for high level expression in the kidney. Promoter and regulatory sequences can be modified in the laboratory to improve the specificity of expression and target expression to specific cell-types in the tissue.

In addition to the promoter sequences discussed above, sequences that regulate transcription in accordance with the present invention are intronic and 3′ regulatory sequences that contain enhancers, splice signals, transcription termination signals and polyadenylation signals, among others. Particularly useful regulatory sequences increase the efficiency of urinary tract-specific expression of proteins in transgenic animals. Especially useful in this regard are the other transcription regulatory sequences of genes expressed at high levels in urinary tract cells, such as the uromodulin gene. Preferred sources for regulatory sequences are rodents (mice and rats), rabbits, poultry, fish, pigs, sheep, goats, cows, horses and humans.

Among the sequences that regulate translation, in addition to the signal sequences discussed above, are ribosome binding sites and sequences that augment the stability of RNA. Especially useful are the translation regulatory sequences of genes expressed at high levels in urinary tract cells. For instance, the urinary tract-specific regulatory sequences of the uromodulin, uroplakin, renin, erythropoietin, uropontin, nephrocalcin, aquaporin genes are preferred, especially those from rodents (mice and rats), rabbits, pigs, poultry, fish, sheep, goat, cows, horses and humans. Particularly preferred are the regulatory sequences of human uromodulin and rat uroplakin genes.

In another aspect of the transgenic animal of the present invention, inducible promoters are preferred, particularly those that can be induced by environmental variables, such as food components. Notable in this regard are metallothionien promoters, which may be induced in animals by incorporating an appropriate metal inducer in feed. Metallothionien promoters have been used to express osteoglycin, epithelin, and bovine oncostatin M in transgenic animals, for instance. Malik et al., Molec. Cell. Biol. 15: 2349-2358 (1995) provides a review of promoters that can be used for tissue-specific or inducible expression or both, and is incorporated by reference herein in its entirety.

It will be appreciated that there may be additional regulatory elements that aid the production of transgenic organisms that express high levels of a protein. Some of these signals may be transcriptional regulators, or signal associated with transport out of the cell. Other signals may play a role in efficient chromosomal integration or stability of the integrated DNA.

Although one aspect of the present invention relates to the expression of proteins that detoxify or degrade organic material, it is understood that other proteins can be expressed in urine according to the methods of the present invention. The cDNAs, genes encoding several human, animal, bacterial, fungal or plant peptides and proteins may be expressed. In particular, enzymes from exthermophilic or thermophilic organisms may be used. Sequences like operons and gene loci coding for related enzymes of a metabolic pathway may be expressed. Specifically, coding sequences for proteins like Prothrombin, Factor VII, Factor IX, Protein C, Protein S, Factor V, Factor VIII, α1-antitrypsin, antithrombin III, fibrinogen, albumin or immunoglobulin may be expressed. Another group of proteins would include hormones and growth factors or cytokines, like growth hormone, erythropoietin, bone morphogenetic proteins, transforming growth factor. Another class of proteins for expression are enzymes like proteases, glycosyltransferases, phosphorylases, kinases, γ-carboxylases, where the protein carries out a posttranslational modification of other proteins like proteolytic processing, glycosylation, phosphorylation, γ-carboxylation. Proteins such as enzyme inhibitors and ion channel proteins may also be expressed. All known and possible mutants, variants or modifications of above listed proteins may also be expressed.

Enzymes involved in the modification of urine, components of urine or its physico-chemical properties and anti-microbial peptides and peptides with bacteriostatic activity also may be expressed.

Enzymatically active peptides or proteins from bacterial, fungal or plant sources that affect the compostion of animal waste. Enzymes that can degrade the penicillin antibiotics from food sources present in excreta will reduce the formation of antibiotic-resistant bacteria and their spread in the environment through sludge. Biologically active peptides or proteins produced in urine could be used to detoxify pesticides contaminating soil and water. Applying known recombinant methods, persons skilled in the art could identify other proteins and genes which could be used in the present invention. For exemplary proteins and genes see FIG. 7. Further, modifications of listed proteins may be expressed, in particular, genetic modifications that allow posttranslational modifications to be performed on the proteins in the host animal. One or several peptides, proteins or enzymes may be produced, and multigenic animals generated that produced several proteins in their urine, thus altering urine composition and that of waste, soil or water. The large volumes of urine or manure generated by said transgenic animals may be used to affect waste management and the degradation of various chemical compounds in the environment.

Thus, in yet another embodiment, the invention relates to an in vivo method of altering a naturally occurring substance in urine. This method involves producing a non-human transgenic animal that produces in its urine a “first” substance that affects a “second” or a “naturally occurring” substance in the urine. By “naturally occurring” is meant that the substance is found in both transgenic and non-transgenic animals of the same species and, therefore, is not expressed by a cloned gene in the transgenic animal. Thus, the substance that alters the naturally occurring substance is expressed by the cloned gene in the non-human transgenic animal. In one embodiment, the first substance is a protein, preferably an enzyme and the second substance is a substrate of such enzyme. For instance, such substrate may be a nitrogen waste product such as guanine, creatine, creatinine, ammonia, urea or uric acid.

### General Methods for Making Transgenic Organisms

Genes may be introduced into an organism in accordance with the invention using standard, well-known techniques for the production of transgenic organisms. These techniques have been the subject of numerous books, including for instance, TRANSGENESIS TECHNIQUES, Murphy et al., Eds., Human Press, Totowa, N.J. (1993), GENETIC ENGINEERING OF ANIMALS, A. Puhler, Ed., VCH Verlagsgesellschaft, Weinheim, N.Y. (1993) and Transgenic Animal Technology, C. A. Pinkert, Ed., Academic Press Inc., San Diego (1994), which are incorporated by reference herein in their entirety.

For instance, DNA can be introduced into totipotent or pluripotent stem cells by microinjection, calcium phosphate mediated precipitation, liposome fusion, retroviral infection or by other means. DNA delivery by electronic pulse into swine oocytes and embryos (Yang et al., Cell Res. 7: 39-49, 1997). Cells containing the heterologous DNA then can be introduced into cell embryos and incorporated therein to form transgenic organisms. Embryonic stem cells and embryonic cell lines for generation of transgenic and chimeric fetuses have been used in mice and pigs (Notarianni et al., Int. J. Dev. Biol. 41: 537-540, 1997).

In a preferred method, developing cells or embryos can be infected with retroviral vectors and transgenic animals can be formed from the infected embryos. In a highly preferred method, DNAs are microinjected into embryos, preferably at the single-cell stage, and the embryos are developed into mature transgenic animals. A step for incubating embryos in vivo or in vitro before transfer to host animals may be added. Yeast artificial chromosome or YAC technology may be used to generate transgenic mice (Schedl et al., Nucl. Acids Res. 20: 3073-3077, 1992), rabbits (Brem et al., Mol. Reprod. Dev. 44: 56-62, 1996) and pigs (Langford et al., Transplant. Proc. 28: 862-863, 1996) containing upwards of 25-500 kb DNA. Entire genomic loci or poly-cistronic operons may be introduced by this method, using pronuclear injection, lipofection into ES cells or yeast spheroblast fusion, among other techniques. Homologous recombination of overlapping DNA fragments in murine zygotes can also be used to generate large, functional transgenes (Pieper et al., Nucl. Acids. Res. 20: 1259-1264, 1992).

In another highly preferred method, nuclei are transferred from cells to embryos and transgenic animals developed from the embryos. Offspring can be derived from embryo transfer in cows (Seidel, G E, J. Dairy Sci. 67: 2786-2796, 1984; Krisher et al., J. Dairy Sci. 78: 1282-1288, 1995; Sims et al., U.S. Pat. No. 5,453,366, 1995) and pigs (Niemann et al., J. Reprod Fertil. 48: 75-94, 1993), and from nuclear transfer in bovine embryos (Robl et al., J. Anim. Sci. 64:642-647, 1987; Maasey, J. M., U.S. Pat. No. 5,057,420, 1991) and of fetal and adult cells in sheep (Wilmut et al., Nature 385: 810-813, 1997).

Transgenic goats (Ebert et al., Bio/Technology 9: 835-838, 1991; Amoah et al., J. Anim. Sci. 75: 578-585, 1997), sheep (Clark et al., Bio/Technology 7: 487-492, 1989), cows (Biery et al., Theriogenology 29: 224, 1988; Krimpenfort et al, Bio/Technology 9: 844-847 1991; Hill et al., Theriogenology 37: 222, 1992; Bowen et al., Theriogenology 39: 194, 1993; Hyttinen et al., Bio/Technology 12: 606-6608, 1994), pigs (Velander et al., Proc Natl. Acad. Sci. 89: 12003-12007, 1992; Wheeler, M. B, U.S. Pat. No. 5,523,226, 1996), rabbits (Buehler et al., Bio/Technology 8: 140-143, 1990), birds, like chicken and quail, fish, like salmon and zebrafish, amphibious lower vertebrates, like Xenopus laevis, invertebrates, like C. elegans and insects like D. melanogaster mice and rats, among others, may be produced with the above technology (Transgenic Animals: Generation and Use, Ed. L. M. Houdebine, Haywood Academic Publishers, The Netherlands, 1997).

Double and other multiply-transgenic animals can be made by introducing two or more different DNAs into the genomic DNA of a multicellular organism using techniques described above. The DNAs may contain the same or different promoters and other expression-regulating sequences. The cDNA or genomic DNAs encoding proteins may be in separate or in single construct. Furthermore, multiply-transgenic organisms also can be made in breeding. For instance, two singly-transgenic organisms can be crossed, using appropriate well known breeding techniques, to generated double-transgenic offspring having the transgenes of both the parents. Successive breeding can be used to introduce additional transgenics as well.

### Organisms In Which Proteins May Be Produced

Non-human multicellular organisms suitable for practicing the invention include plants and animals. Animals include invertebrates and vertebrates, like birds, reptiles, insects, fish and mammals. Particularly preferred are mammals, other than humans, for producing substances in urine. Preferred mammals include mice, rats, hamsters, guinea pigs, rabbits, cats, dogs, pigs, sheep, goats, cows and horses. Among livestock animals, cows, goats, sheep and pigs are preferred, among research animals are the foregoing and dogs, cats, hamsters, rabbits, rats and mice. Among birds, chickens, ducks, and turkeys are preferred.

### Cells, Tissues, Fluids and Other Compartments for Expression

Generally, any cell or tissue of an organism may be used in accordance with the present invention. Preferred, in this regard, are cells and tissues that secrete substances into bodily fluids. These cells may be used as a source of nuclei for nuclear transfer to produced transgenic animals. Isolated cells may be grown in culture using standard mammalian tissue culture methods. In this regard, cells and tissues that secrete peptides and proteins into urine are highly preferred. Among these, proximal tubule and bladder epithelial cells that secrete proteins into urine are especially preferred.

### Illustrative Products

It will be appreciated that the invention can be used to produce a peptide or protein with enzymatic activity in a cell to influence the production of non-proteinaceous, as well as proteinaceous, products of cell metabolism and catabolism. The targeted expression of heterologous proteins to the kidney or other parts of the urinary tract will result in the alteration of the composition of urine, due to the added presence of the foreign protein, as well as due to modification of urine components and their effects.

It should be understood, however, that the above detailed description and the following specific examples, while indicating preferred embodiments of the invention, are given by way of illustration only, since various changes and modifications within the spirit and scope of the invention will become apparent to those skilled in the art from this detailed description.

### EXAMPLES

The following examples describe the production of a complex human protein, HPC, in the urine of transgenic animals.

### Example 1

(A) Construction of WAP/HPC Transgene and Generation of Mice and Pigs.

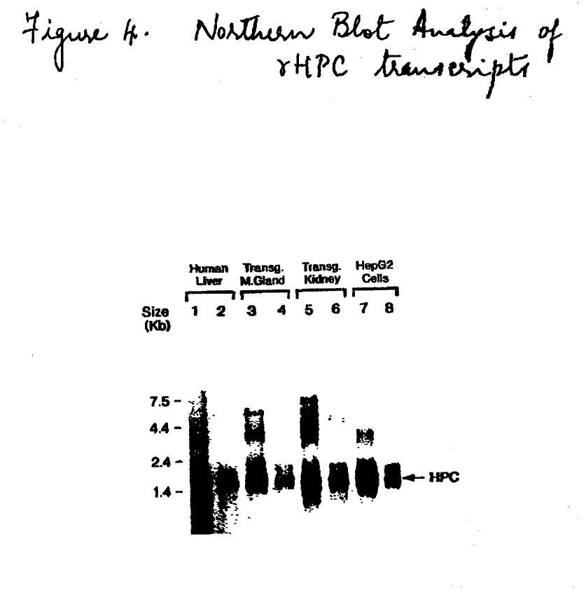

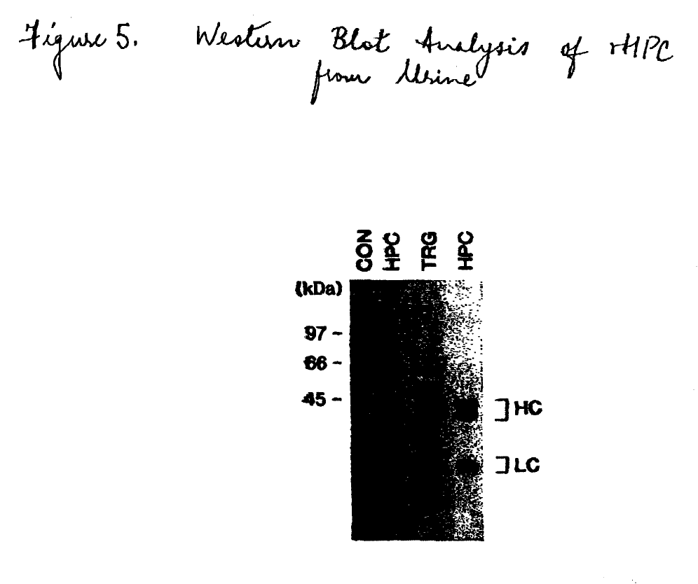

Transgenic mice and pigs were produced containing a transgene composed of a murine whey acidic protein promoter and the human Protein C (HPC) gene. Transgenic pigs containing a transgene composed of the HPC cDNA inserted into the mouse whey acidic protein gene were generated (Velander et al., Proc Natl Acd. Sci, 89: 12003-12007, 1992). The promoter is well known and has been used to direct expression and secretion of rHPC into milk in transgenic mammals, as described in, for instance, Paleyanda et al., Transgenic Res. 3: 355-343 (1994), which is incorporated by reference herein in its entirety. The DNA construct comprised a 4.1 kb mouse whey acidic protein (WAP) promoter and a 9 kb HPC gene with 0.4 kb 3′ nontranslated sequences (FIG. 3A). It was constructed from readily available DNAs using well-known techniques, as described in Drohan et al., Transgenic Res. 3: 355-364 (1994) and Hogan et al., MANIPULATING THE MOUSE EMBRYO, Cold Spring Harbor Press (1986), each of which is incorporated by reference herein in its entirety.

HPC circulates in plasma as a 62 kDa zymogen and activated HPC has potent anticoagulant activity (FIG. 3B). The 19 amino acid signal peptide directs translocation of the nascent polypeptide into the hepatocyte endoplasmic reticulum (ER) and is cleaved by a signal peptidase. The 24 residue propeptide mediates the binding of vitamin K-dependent (VKD) γ-glutamyl carboxylase, an integral ER membrane protein. The carboxylase utilizes reduced vitamin K, CO₂ and O₂ to convert nine Glu residues to γ-carboxyglutamic acid (Gla), following the addition of the glycosyl core. The Gla domain is essential for Ca⁺²-mediated activation of the zymogen, binding to phospholipids, thrombin-thrombomodulin, platelet factor 4 and for plasminogen activator inhibitor inactivation. In its transit through the Golgi, complex carbohydrates are added to four N-linked sites, the propeptide removed and an internal KR dipeptide cleaved to generate a light and a heavy chain held together by a disulfide bond. HPC undergoes β-hydroxylation through the action of the aspartyl β-hydroxylase at Asn residues in the epidermal growth factor-like (EGF) domain, which also binds Ca+2. After secretion, the activation peptide is proteolytically cleaved by thrombin to generate activated HPC. The heavy chain contains the serine protease domain and is implicated in multiple roles, such as mononuclear phagocyte response, α₂-macroglobulin binding, inactivation of plasminogen activator inhibitor and inhibition of cytokine production by monocytes.

(B) Detection of Transgene Expression in the Kidney and of rHPC in Urine

Total RNA was prepared from tissues of transgenic females of the F1 or F2 generations and from control mice using standard techniques. RNA was isolated from fresh or frozen tissues in a single step procedure using acid guanidinium thiocyanate phenol-chloroform extraction (available commercially, for instance, as RNAzol., Molecular Research Center, Inc. and described in Chomczynski et al., Anal. Biochem. 162: 156-159 (1987), which is incorporated herein by reference in its entirety.

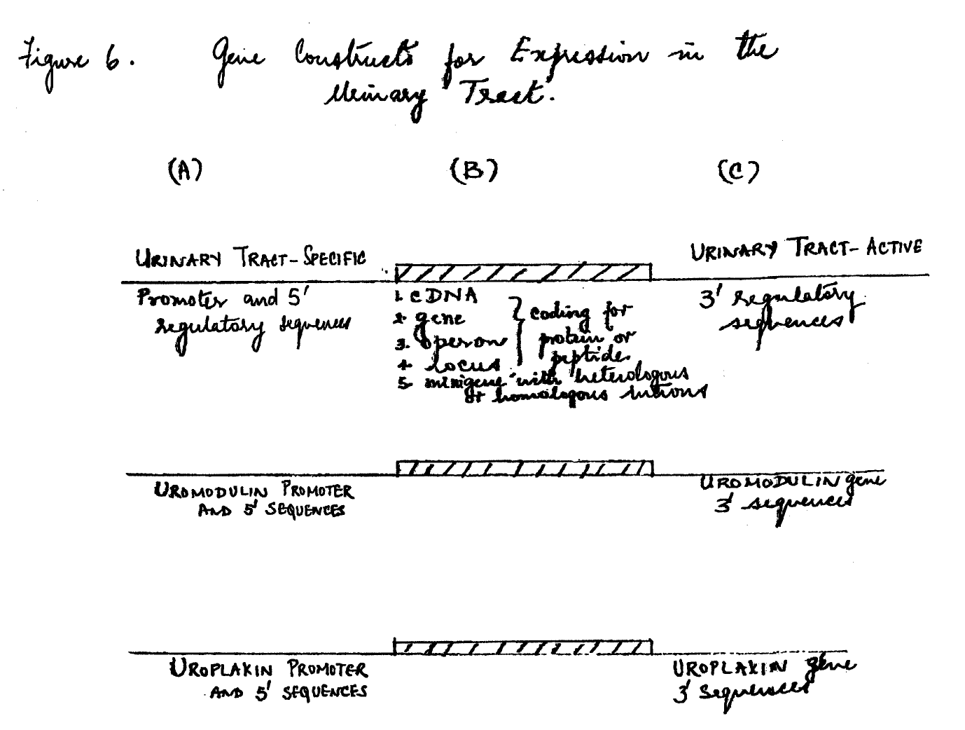

The transgenic animals expressed HPC transgene in the mammary gland and secreted recombinant human PC (rHPC) into milk. The transgene was expressed to a lower extent in the kidney as detected by Northern blotting of total RNA from the kidneys of transgenic mice (FIG. 4). Northern blot analysis of total RNA (1, 3, 5, 7) and mRNA (2, 4, 6, 8) from tissues of mice transgenic for HPC was carried out. Transcripts from human liver (lanes 1-2), the mammary gland (lanes 3-4) and kidney (lanes 5-6) of WAP/HPC transgenic mouse 4.2.10.9 (Drohan et al., Transgenic Res. 3: 355-364 (1994) and human liver HepG2 cells (lanes 7-8) were analyzed. To obtain signals of similar intensity, different amounts of RNA were loaded in lanes 1 through 8; 3.7, 0.11, 0.004, 0.0001, 3.7, 0.096, 2.1 and 0.021 μgs respectively. Blots were hybridized with HPC cDNA probes as in (Drews et al., Proc. Natl. Acad. Sci. 92: 10462-10466 1994). The arrow indicates the mature rHPC transcript, RNA standards in kilobases are given on the left.

Thus, any rHPC protein detected in urine comes from its synthesis in the kidney and not from the circulating plasma. As a result of this observation, the urine of these transgenic animals was assayed by ELISA for the presence of rHPC. Urine was collected from transgenic mice and dialyzed. A sandwich ELISA performed on the urine of WAP/HPC transgenic mice resulted in the detection of 64-76 ng/ml of rHPC. This was confirmed in pigs where rHPC expression levels were high enough to allow detection by ELISA, western blotting and isolation by immunoaffinity chromatography. The ELISA was carried out by coating 96-well microtiter plates with rabbit anti-HPC polyclonal antibodies. A standard curve was constructed using HPC at concentrations from 1.3 to 42.5 ng/ml. Dialyzed urine samples were diluted 10, 20, 40 and 80-fold to raise the level of detection. Urine from pig 110-3 was diluted more, as it contained more rHPC. Dilutions of the plasma-derived HPC standard were also incubated for 20 minutes at 37 degrees celsius. Excess, unbound antigen was removed by washing plates and then incubating them with a 1:1000 dilution of goat anti-HPC antibody, followed by a 1:1000 dilution of HRP-labelled anti-goat IgG. The OPD substrate was added for 3 mins, the reaction stopped with 3N sulphuric acid and plates read at 490 nm. Transgene expression in the kidney was connected with protein synthesis, as rHPC was detected in the urine of both mice and pigs (Table III). Thus, the urine of livestock animals can be used as a body fluid for the production of large amounts of peptides and proteins.

TABLE III

| | rHPC in urine |
|---|---|
| | (polyclonal ELISA, |
| Animal | μg/ml) |
| cDNA Mice | 0.064-0.076 |
| cDNA Pigs | |
| line 29-2, #115-6 | 0.10-0.90 |
| line 83-1, #83.1 | 0.14-0.26 |
| Genomic Pigs | |
| line 110-3, #110- | 0.58-18.0 |
| 3 | 0.11-0.74 |
| #122-5 | 0.05-0.18 |
| #122-6 | 0.02-0.20 |
| line 110-1, #110- | 0.11-0.29 |
| 1 | 0.08-0.26 |
| #114-6 | |
| #114-7 | 0.18-0.36 |
| line 9-7, #9-7 | |
| Control Pigs | not detected |

(C) Detection of Activity of rHPC

To determine if secreted rHPC in urine had activity, rHPC was enriched from the urine of a transgenic sow and subjected to activity assays. Transgenic pig urine was dialyzed against 20 mM sodium citrate, 80 mM sodium chloride, pH 6.5, then diluted 50% in this buffer. rHPC was enriched from the urine of transgenic sow 110-3 using an antibody to the heavy chain of HPC, 8861 MAb, coupled to Azalactone. 500 ml of diluted urine containing about 5.2±0.8 μg/ml rHPC was loaded at 1 cm/min. The column was washed in 20 mM sodium citrate, 80 mM sodium chloride, pH 6.5. Fractions were eluted with 100 mM sodium carbonate, pH 10 and with 2 M sodium thiocyanate. About 32% of rHPC loaded was recovered in the 2 M sodium thiocyanate fractions.

Enriched fractions were analyzed for activity in chromogenic assays (Table IV), as described in Drohan et al., Transgenic Res. 3: 355-364 (1994). An HPC standard isolated from human plasma by immunoaffinity purification using the same monoclonal antibody was employed as a reference in the assays. The activity of HPC was considered to be 100% in the assays. Results are given below.

TABLE IV

| | Concentration of | Amidolytic activity |
|---|---|---|
| Source of | protein in enriched | of protein from |
| rHPC | fractions (ELISA) | enriched fractions |
| Pig urine | 400 ± 30 μg/ml | 31 ± 4 U/mg |

Thus the urinary tract cells can produce not only endogenous proteins, but also complex foreign proteins that retain activity.

(D) Western Blot Detection of Processed HPC

The size and processing of rHPC from urine was analyzed. Immunoaffinity chromatography using the 8861 MAb against the heavy chain of HPC was employed to enrich rHPC from the urine of 6 month old sow 110-3. Proteins eluted using 2 M sodium thiocyanate from the column were analyzed. Urinary proteins from control and transgenic pigs were resolved by 10% SDS-PAGE under reducing conditions and western blotted (FIG. 5). Blots were probed with a rabbit anti-HPC polyclonal antibody, detected with goat anti-rabbit antibodies conjugated to HRP and developed with 4-cloronaphthol substrate. The presence of rHPC in the urine altered its composition, but the rHPC itself was posttranslationally processed into the heavy and light chains as in plasma HPC. The molecular weight of the rHPC forms from urine appeared to be similar, indicating that glycosylation had also occurred in the kidney cells.

It is clear from the above that the gene for a complex human protein like HPC was expressed in the urinary tract of mice and pigs and the 66 Kda protein was produced in urine. RHPC was well processed to the mature forms of 44 Kda heavy and 22 Kda light chains. The enriched protein retained functional activity. Thus, animal urinary tract cells can produce complex foreign proteins that retain activity.

### Example 2

Isolation of the human Uromodulin (THP) promoter A human genomic library constructed in P1 bacteriophage (Sternberg, N. L., Trends Genet. 8:11-16, 1992) was screened by polymerase chain reaction using sequences located in the 5′ region of the uromodulin cDNA (Pennica et al., Science 236: 83-87, 1987). P1 plasmids present in cre+E. coli hosts can contain genomic DNA inserts of 75-100 kb. The sequence of oligonucleotides oligos used in screening are given below: #4683 (3′ primer) CCC AGG CTC AGC GCA CTC ATC C #4684 (5′ primer) GTC ACA GCA ATG CCA CCT GAC The oligos were synthesized, precipitated and resuspended in Tris-EDTA buffer before PCR. Three P1 clones were isolated for subcloning of 5′ flanking sequences into pBluescript plasmid.

Likewise, the promoters of other urinary tract-specific genes may be isolated, particularly as a result of human and animal genome sequencing projects.

### Example 3

### Gene Constructs for Expression in the Urinary Tract

General constructs for the expression of complex peptides and proteins in the urinary tract of transgenic animals will include:

(A) 5′ expression regulating sequences, including urinary tract-specific promoter and enhancer sequences; (B) cDNA or genomic DNA sequences encoding complex peptides and proteins with enzymatic activity, and a signal sequence effective in directing the secretion of said peptide or protein into the urine of transgenic animal; and (C) 3′ regulatory sequences, including a polyadenylation sequence, that results in the expression of said DNA sequences in the urinary tract cells; wherein A, B and C are operably linked in said gene construct to obtain the production of said peptide or protein in urinary tract cells and secretion into urine of animal.

Non-exclusive examples of (A) and (C) from genes of:

Uromodulin

Uroplakin

Renin

Erythropoietin

Apolipoprotein E

Aquaporin

Nephrocalcin

Osteopontin-k/Uropontin

Urinary Kallikrein

Urinary Thrombomodulin

Non-exclusive examples of (B) from cDNA and genes of proteins listed in FIG. 7.

All citations to journals, books, patents and applications set forth above are herein incorporated by reference, in pertinent part or in their entirety.

What is claimed is:

1. A method of producing a protein in urine that degrades or detoxifies organic material, said method comprising:
(a) providing a non-human transgenic animal having stably integrated into its genome an exogenous gene encoding a protein that is detectable in urine and that degrades or detoxifies organic material.

2. The method of claim 1, wherein said transgenic animal is a mammal.

3. The method of claim 1, wherein said transgenic animal is selected from the group consisting of a pig, sheep, goat, cattle, rodent, rabbit, horse, dog, cat, bird and reptile.

4. The method of claim 1, wherein said protein is an enzyme.

5. The method of claim 4, wherein said enzyme is selected from the group consisting of the list of enzymes in FIG. 7.

6. The method of claim 1, wherein said organic material is feces, urine, microbe, a chemical pollutant or a by-product thereof, and a food product or a by-product thereof.

7. The method of claim 6, wherein said chemical pollutant is selected from the group consisting of an herbicide, pesticide and fertilizer.

8. A non-human transgenic animal that produces in its urine a protein that degrades or detoxifies an organic material, wherein said non-human transgenic animal has stably integrated into its genome an exogenous gene encoding a protein that degrades or detoxifies an organic material and that is detectable in urine.

9. The transgenic animal of claim 8, wherein said transgenic animal is a mammal.

10. The transgenic animal of claim 8, wherein said transgenic animal is selected from the group consisting of a pig, sheep, goat, cattle, rodent, rabbit, horse, dog, cat, bird and reptile.

11. The transgenic animal of claim 8, wherein said organic material is feces, urine, microbe, a chemical pollutant or a by-product thereof, and a food product or a by-product thereof.

12. The transgenic animal of claim 8, wherein said organic material is produced by said transgenic animal or by a different animal.

13. The transgenic animal of claim 11, wherein said chemical pollutant is selected from the group consisting of an herbicide, pesticide and fertilizer.

14. The transgenic animal of claim 8, wherein said protein is an enzyme.

15. The transgenic animal of claim 14, wherein said enzyme is selected from the group of enzymes listed in FIG. 7.

16. A method of degrading or detoxifying organic material, comprising the steps of:
(a) providing a non-human transgenic animal having stably integrated into its genome an exogenous gene encoding a protein that is detectable in urine and that degrades or detoxifies organic material; and
(b) contacting said organic material with said urine, thereby degrading and detoxifying said organic material.

17. The method of claim 16, wherein said contacting involves mixing said urine with said organic material.

18. The method of claim 16, wherein said contacting involves said non-human transgenic animal urinating on said organic waste.

19. The method of claim 16, wherein said transgenic animal is a mammal.

20. The method of claim 16, wherein said transgenic animal is selected from the group consisting of a pig, sheep, goat, cattle, rodent, rabbit, horse, dog, cat, bird and reptile.

21. The method of claim 16, wherein said organic material is selected from the group consisting of feces, urine, microbe, a chemical pollutant or a by-product thereof, and a food product or a by-product thereof.

22. The method of claim 21, wherein said chemical pollutant is selected from the group consisting of an herbicide, pesticide and fertilizer.

23. The method of claim 16, wherein said protein is an enzyme.

24. The method of claim 23, wherein said enzyme is selected from the group consisting of the enzymes listed in FIG. 7.

25. A facility for containing animals, said facility comprising:
(a) at least one non-human transgenic animal having stably integrated into its genome an exogenous gene encoding a protein that is detectable in urine and that degrades or detoxifies organic material; and
(b) a structure for containing said animal within said facility.

26. The facility of claim 25, further comprising:
(c) at least one non-transgenic animal of the same or different species from said transgenic animal.

27. The facility of claim 25, wherein said transgenic animal is a mammal.

28. The facility of claim 25, wherein said transgenic animal is selected from the group consisting of a pig, sheep, goat, cattle, rodent, rabbit, horse, dog, cat, bird and reptile.

29. The facility of claim 25, wherein said transgenic animal is a mammal and wherein said non-transgenic animal is also an mammal.

30. The facility of claim 27, wherein said non-transgenic animal is a bird or reptile.

31. The facility of claim 25, which is selected from the group consisting of a farm, ranch, slaughter house, research facility and zoo.

32. The method of claim 25, wherein said organic material is selected from the group consisting of feces, urine, microbe, a chemical pollutant and a by-product thereof and a food product and a by-product thereof.

33. The method of claim 32, wherein said chemical pollutant is selected from the group consisting of an herbicide, pesticide and fertilizer.

34. An in vivo method of altering a substance in urine, said method comprising:
(a) producing a non-human transgenic animal having stably integrated into its genome an exogenous gene encoding a a first substance that alters a second substance in the urine of said transgenic animal.

35. The method of claim 34, wherein said animal is a mammal.

36. The method of claim 34, wherein said transgenic animal is selected from the group consisting of a pig, sheep, goat, cattle, rodent, rabbit, horse, dog, cat, bird and reptile.

37. The method of claim 36, wherein said mammal is a pig.

38. The method of claim 34, wherein said first substance degrades said second substance.

39. The method of claim 38, wherein said first substance is a protein.

40. The method of claim 39, wherein said protein is an enzyme.

41. The method of claim 40, wherein said enzyme is selected from the group consisting of the enzymes listed in FIG. 7.

42. A DNA construct for the production in the urine of a non-human transgenic animal, of a protein that degrades or detoxifies organic material, said construct comprising
(a) 5′ expression regulating sequences, including urinary tract-specific promoter and enhancer sequences;
(b) cDNA or genomic DNA sequences encoding said protein, and a signal sequence effective in directing the secretion of said protein into the urine of transgenic animal; and
(c) 3′ regulatory sequences, including a polyadenylation sequence, that results in the expression of said DNA sequences in urinary tract cells;
wherein a, b and c are operably linked in said DNA construct to obtain the production of said protein in said urine of said animal.

43. The DNA construct of claim 42, wherein (a) and/or (c) are sequences from the gene encoding a protein selected from the group consisting of uromodulin, uroplakin, renin, erythropoietin, apolipoprotein E, aquaporin, nephrocalcin, osteopontin-k, uropontin and urinary kallikrein.

44. The DNA of claim 42, wherein the protein of (b) is selected from the group consisting of the enzymes listed in FIG. 7.
